(19) United States
(12) Patent Application Publication
(10) Pub. No.: US 2025/0218577 A1
(43) Pub. Date: Jul. 3, 2025

(54) SYSTEMS AND METHODS FOR DETERMINING UNNECESSARY INTERNAL SYSTEM UTILIZATION

(71) Applicant: Optum, Inc., Minnetonka, MN (US)

(72) Inventors: Yao ZHANG, La Canada Flintridge, CA (US); James M. DOLSTAD, Leavenworth, WA (US); Aparna PHILKANA, Naples, FL (US); Maxine GOLDSMITH, West Hartford, CT (US); Rajiv ARYA, Skillman, NJ (US)

(21) Appl. No.: 18/400,032

(22) Filed: Dec. 29, 2023

(51) Int. Cl. G16H 40/20

(52) U.S. Cl. CPC G16H 40/20

(57) ABSTRACT

Systems and methods are disclosed for determining unnecessary internal system utilization. A method includes receiving a first data object and generating an entity data object for each entity of the plurality of entities based on at least a portion of the first data object. The method further includes generating a usage indicator for each entity of the plurality of entities by applying a machine-learning model to at least a portion of the entity data object for the entity, the usage indicator associated with a pre-determined time period. The method further includes generating a utilization data object based on the entity data object and the usage indicator generated for each entity, and causing the utilization data object to be displayed on a Graphical User Interface (GUI).

### TECHNICAL FIELD

The present disclosure generally relates to the field of data analytics. In particular, the present disclosure relates to systems and methods for modeling and predicting unnecessary internal system utilization based on analyzing various data sources.

### BACKGROUND

Ambulatory Care Sensitive Conditions (ACSCs) are conditions for which appropriate external entity management and interventions can prevent complications, more severe conditions, or the need for internal system utilization. Management of these conditions in an external setting is a key factor in reducing unnecessary internal system utilization. Current techniques include entity education, entity assistance coordination, and regular external follow-up measures. However, these techniques suffer from one or more issues and may be improved in one or more ways.

For instance, current techniques often struggle to provide personalized, comprehensive entity management that addresses the unique needs and circumstances of each entity. Entity education is a part of external management, but broad, non-specific education efforts may not effectively reach or influence individual entities. Entity assistance coordination is critical but can be hindered by communication gaps or latency between involved systems or sectors. Regular external follow-up measures are important, yet adherence can be influenced by numerous factors. Current predictive models may not fully capture the complex, multifaceted nature of ACSC risk, and may not properly identify condition severity levels for adequate intervention measures. The consequence of these shortcomings includes an increased unnecessary internal system utilization, which could have been prevented through appropriate external entity management and interventions.

Therefore, there is a need for a more sophisticated and accurate approach to predicting and mitigating unnecessary internal system utilizations, resulting from mismanaging externally-preventable conditions.

This disclosure is directed to addressing the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section

### SUMMARY

The present disclosure addresses the technical problem(s) described above or elsewhere in the present disclosure and improves the state of conventional healthcare management techniques.

In some aspects, the techniques described herein relate to a computer-implemented method including: receiving, by one or more processors, a first data object, the first data object including: an entity data set containing a plurality of entities; a utilization event data set containing a plurality of utilization event records; an event data set; an environmental data set; and a performance metric data set; generating, by the one or more processors, an entity data object for each entity of the plurality of entities based on at least one of the entity data set, the utilization event data set, the event data set, the environmental data set, or the performance metric data set; generating, by the one or more processors, a usage indicator for each entity of the plurality of entities by applying a machine-learning model to at least a portion of the entity data object for the entity, the usage indicator associated with a pre-determined time period; generating, by the one or more processors, a utilization data object based on the entity data object and the usage indicator generated for each entity; and causing, by the one or more processors, the utilization data object to be displayed on a Graphical User Interface (GUI).

In some aspects, the techniques described herein relate to a system including memory and one or more processors communicatively coupled to the memory, the one or more processors configured to: receive a first data object, the first data object including: an entity data set containing a plurality of entities; a utilization event data set containing a plurality of utilization event records; an event data set; an environmental data set; and a performance metric data set; generate an entity data object for each entity of the plurality of entities based on at least one of the entity data set, the utilization event data set, the event data set, the environmental data set, or the performance metric data set; generate a usage indicator for each entity of the plurality of entities by applying a machine-learning model to at least a portion of the entity data object for the entity, the usage indicator associated with a pre-determined time period; generate a utilization data object based on the entity data object and the usage indicator generated for each entity; and cause the utilization data object to be displayed on a Graphical User Interface (GUI).

In some aspects, the techniques described herein relate to one or more non-transitory computer-readable storage media including instructions that, when executed by one or more processors, cause the one or more processors to: receive a first data object, the first data object including: an entity data set containing a plurality of entities; a utilization event data set containing a plurality of utilization event records; an event data set; an environmental data set; and a performance metric data set; generate an entity data object for each entity of the plurality of entities based on at least one of the entity data set, the utilization event data set, the event data set, the environmental data set, or the performance metric data set; generate a usage indicator for each entity of the plurality of entities by applying a machine-learning model to at least a portion of the entity data object for the entity, the usage indicator associated with a pre-determined time period; generate a utilization data object based on the entity data object and the usage indicator generated for each entity; and cause the utilization data object to be displayed on a Graphical User Interface (GUI).

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1A is a diagram showing an example of a system configured for healthcare management, according to some embodiments of the disclosure.

FIG. 1B is a diagram of example components of a value impact platform, according to some embodiments of the disclosure.

FIG. 1C is a diagram of example components of a healthcare management module, according to some embodiments of the disclosure.

FIG. 2 is a flowchart showing a method for determining unnecessary internal system utilization, according to some embodiments of the disclosure.

FIG. 3 shows an example machine-learning training flow chart, according to some embodiments of the disclosure.

FIG. 4 illustrates an implementation of a computer system that executes techniques presented herein, according to some embodiments of the disclosure.

### DETAILED DESCRIPTION

The present disclosure relates to the field of data analytics and artificial intelligence. Various embodiments of this disclosure relate generally to techniques for predicting unnecessary internal system resource utilization, and, more particularly, to systems and methods for modeling predicted unnecessary internal system resource utilization and interventions to increase efficiency of resource utilization.

As previously discussed, current methods for managing Ambulatory Care Sensitive Conditions often fall short in delivering personalized entity management, addressing communication lapses, ensuring consistent adherence to external follow-ups, and utilizing accurate predictive models to capture the intricate nature of ACSC risk.

To address these concerns, a centralized system and method are provided which facilitate the comprehensive monitoring, analysis, and optimization of external entity management and interventions. This system adeptly integrates multiple data sets, combining various attributes, events, and performance metrics of the entities. By employing advanced analytical methodologies, such as machine-learning algorithms, the system is adept at identifying patterns and correlations that suggest inefficient and/or unnecessary internal system resource allocation or utilization. Furthermore, these analyses not only provide insights but also actionable recommendations to improve the efficiency of internal system resource distribution and utilization. Moreover, the systems and methods described herein leverage data that is unique to individual entities and addresses potential entity interventions at the entity-level. The system and method further include monitoring of the entity data and its changes over time, adjusting, updating, and retraining the applied models to account for changes in entity data, resulting in higher adoption of interventions, improved care pathways for the entities, and reduced unnecessary internal system resource utilization. The above technical improvements, and additional technical improvements, will be described in detail throughout the present disclosure. Also, it should be apparent to a person of ordinary skill in the art that the technical improvements of the embodiments provided by the present disclosure are not limited to those explicitly discussed herein, and that additional technical improvements exist.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

Various non-limiting embodiments of the present disclosure will now be described to provide an overall understanding of the principles of the structure, function, and use of systems and methods disclosed herein for healthcare management outcomes.

Reference to any particular activity is provided in this disclosure only for convenience and not intended to limit the disclosure. A person of ordinary skill in the art would recognize that the concepts underlying the disclosed devices and methods may be utilized in any suitable activity. For example, while the present disclosure is in the context of healthcare management, one of ordinary skill would understand the applicability of the described systems and methods to similar tasks in a variety of contexts or environments. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Training the machine-learning model may include one or more machine-learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. After training the machine-learning mode, the machine-learning model may be deployed in a computer application for use on new input data that it has not been trained on previously.

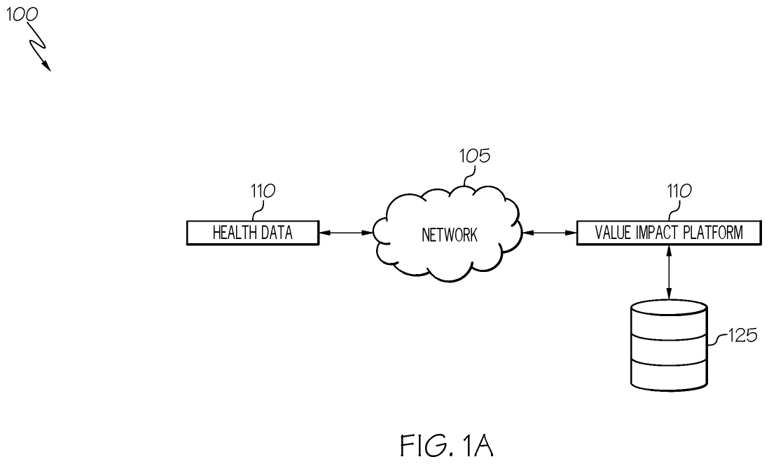

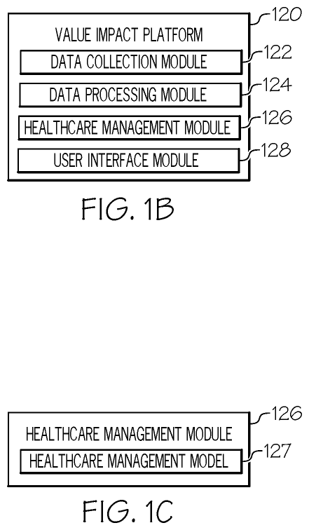

FIG. 1A is a diagram showing an example of a system that is capable of healthcare management, according to some embodiments of the disclosure. The depicted network environment, designated as 100, is in accordance with a specific embodiment of the current disclosure. The network environment 100 encompasses a communication infrastructure, such as network 105, which is accompanied by health data 110, and is further equipped with a value impact platform 120 integrated with a database 125.

In some embodiments, various components of the network environment 100 interact with each other through the network 105. The network 105 facilitates communication between the value impact platform 120 and one or more other systems, including one or more data sets, such as (but not limited to) health data 110. The one or more data sets and/or health data 110 includes data, one or more data entries, and/or data objects associated with or comprising medical records. The network 105 includes one or more networks such as a data network, a wireless network, a telephony network, or any combination thereof.

The health data 110 encompasses an array of structured and unstructured information pertaining to the health of individuals. The health data, in some embodiments, is in the form of one or more data objects, and encompass various facets, including but not limited to, health plan-provider contracts, member files, provider records, PCP to member attribution, medical and pharmacy claims, as well as insights from Impact Analytics, geographical and context based pricing indexes, Social Determinants of Health (SDoH), NYU Avoidable Preventable classification, Admit, Discharge, Transfers (ADT), Area Deprivation Index (ADI), Rural Urban (RUCA), risk and quality analytics, and the like. This diverse health data repository, comprising details such as demographic data, medical histories, insurance claims, and other health metrics, finds its repository in storage, which may take the form of local or remote data storage solutions, including file servers and cloud-based storage systems, among others.

The database 125 is used to support the storage and retrieval of data related to one or more data sets and/or data objects, such as the health data 110, storing metadata and/or healthcare data about one or more populations represented in the health data 110, as well as any information received from the value impact platform 120. The database 125 can consist of one or more systems, such as a relational database management system (RDBMS), a NoSQL database, or a graph database, depending on the requirements and use cases of the network environment 100.

In some embodiments, the database 125 is any type of database, such as relational, hierarchical, object-oriented, etc., wherein data is organized in tables, lookup tables, or other suitable manners. The database 125 stores and provides access to data utilized by the value impact platform 120. The database 125 stores information related to the health data 110 as well as information generated by the value impact platform 120. The database 125 can store various types of information to aid in the healthcare management.

In some embodiments, the database 125 includes a machine learning-based training database that maps relationships, associations, connections, or the like between input parameters from the health data 110 and output parameters representing the one or more metrics for management of healthcare. For example, the training database can include machine learning algorithms that learn mappings between medical data inputs and one or more of utilization, adherence, or sensitive condition treatment outputs. The training database can be routinely updated based on additional machine learning.

The value impact platform 120 communicates with other components of the network 105 using known or developing protocols. These protocols govern interactions between network nodes and define rules for generating, receiving, and interpreting information sent over communication links. The protocols operate at different layers, from generating physical signals to identifying software applications sending or receiving the information.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers.

In operation, the network environment 100 provides a framework for analyzing large amounts of health data 110, leveraging data analytics, artificial intelligence, and database technologies to support various use cases and applications. For example, the network environment 100 can be used to generate metrics, data objects, and insights from one or more data sets, such as the health data 110, based on user-defined criteria or a plurality of parameters.

To perform these tasks, the value impact platform 120 utilizes techniques such as the healthcare management model 127 (FIG. 1C), which analyzes the health data 110 and identifies one or more healthcare management metrics, which in some embodiments match one or more specified criteria. The value impact platform 120 can also utilize the data collection module 122 and data processing module 124 (FIG. 1B) to gather and prepare the health data 110.

To support storage and retrieval of data related to the healthcare management metrics, the database 125 stores metadata about the health data 110, such as data sources, types, and formats. The database 125 also stores information about the health management metrics output by the value impact platform 120, such as health criteria, identifiers, and statistics.

In addition to healthcare management, the network environment 100 can support other applications like data visualization, search, and predictive modeling. For example, the network environment 100 could allow users using user devices to search the health data 110 for one or more metrics matching certain criteria, or visualize healthcare metric statistics through interactive graphs and charts.

FIG. 1B is a diagram of example components of a value impact platform 120, according to some embodiments of the disclosure. Referring to FIG. 1B, the value impact platform 120 is a component of the network environment 100. The value impact platform 120 provides the capabilities to analyze one or more data sets, such as health data 110 and generate one or more data objects including one or more healthcare management metrics. As used herein, terms like “component” or “module” encompass hardware and/or software implemented by a processor or the like. For example, the value impact platform 120 includes components for collecting, processing, and analyzing health data as well as generating one or more data objects including one or more healthcare management metrics. To that end, the value impact platform 120 includes modules such as a data collection module 122, a data processing module 124, a healthcare management module 126, and a user interface module 128. It is contemplated that the functions of these modules could be combined into fewer modules or performed by other modules with equivalent functionality.

In some embodiments, the data collection module 122 of the value impact platform 120 undertakes the collection of data from one or more data sets, such as health data 110, during the operation of the environment 100. The data collection module 122 is equipped to receive a myriad of data types such as, but not limited to, health plan provider contract data, provider data, member data, PCP-to-member attribution data, medical and pharmacy claims data, proprietary or generated data, such as impact analytics data, pricing data, risk and quality analytics data, or the like, Healthcare Effectiveness Data and Information Set (HEDIS) quality metrics data, clinical prediction analytics, financial savings factors, Social Determinants of Health (SDoH) data, NYU Avoidable Preventable classification data, Area Deprivation Index (ADI) data, Admit, Discharge, and Transfer (ADT) data, Rural-urban Commuting Area (RUCA) data, proprietary episode treatment groupers (ETGs) data, proprietary service categories data, AHRQ groupers data, member geographic data, and the like.

In some embodiments, the health plan provider contract data includes, but is not limited to, the identification and credentials of providers, specifics of the health plans offered, a compilation of service and billing codes, agreed reimbursement rates, payment terms, the scope of benefit coverage, eligibility prerequisites for patients, protocols for authorizations and referrals, quality and performance benchmarks, procedures for dispute resolution, duration and termination information, privacy and confidentiality terms, regulatory adherence protocols, amendment procedures for the contract, utilization review guidelines, potential risk-sharing agreements, credentialing processes for healthcare providers, specifications regarding pharmacy formularies, and the like.

In some embodiments, the provider data includes, but is not limited to, identifiers such as names, addresses, contact details, specialties, qualifications, and tax identification numbers associated with providers. The data set also includes credentialing information, which verifies the qualifications and backgrounds of the providers, their affiliations with hospitals or other medical institutions, the insurance plans they accept, and their availability for patient appointments. In addition, the provider data contains historical data on the types and volumes of procedures performed, quality of care metrics, patient outcomes, and satisfaction scores, as well as data on billing practices and reimbursement rates.

In some embodiments, the member data includes, but is not limited to, identifiers such as names, birth dates, and member identification numbers associated with members. It further contains demographic details like addresses, contact information, gender, and employment information if relevant to the health plan. The health-related aspects of the data set cover a member's entire medical history with the plan, including plan enrollment dates, coverage details, dependents, benefit utilization records, and claims history. Additionally, it includes members' health conditions, diagnoses, treatment histories, and outcomes.

In some embodiments, the PCP-to-member attribution data includes, but is not limited to, one or more mappings between primary care providers (PCPs) and their attributed members, thereby identifying and/or linking individuals enrolled in a health plan and their designated primary caregivers. It includes data related to member identification numbers, names, and demographic information, alongside corresponding identifiers and credentials of the attributed PCPs. The PCP-to-member attribution data includes the duration of the member-PCP relationship, visit histories, and the nature of primary care services rendered. Additionally, in some embodiments, it includes data on care continuity, referral patterns, and the effectiveness of the PCP in managing the member's health, including preventative care and chronic disease management.

In some embodiments, medical and pharmacy claims data includes, but is not limited to, comprehensive records of members' interactions with healthcare systems, reflecting services rendered and pharmaceuticals provided. This includes data on claims submissions, detailing dates of service, types of services, service providers, claim amounts, and payment outcomes. Each entry correlates with member identification numbers and the associated healthcare providers or pharmacies. The medical and pharmacy claims also includes diagnostic codes, procedure codes, and pharmacy billing information, providing insights into the medical conditions treated and the medications dispensed. Furthermore, in some embodiments, the medical and pharmacy claims includes a historical overview of members' claims over time, which can be analyzed to ascertain patterns in healthcare utilization, medication adherence, and the overall efficiency of healthcare services delivered.

In some embodiments, impact analytics data, such as data generated from a proprietary analytics engine, includes but is not limited to, one or more data sets which provide insights and/or data related to healthcare efficiency, costs, and outcomes. The impact analytics data aggregates and analyzes various aspects of healthcare services, encompassing medical claims, pharmacy claims, clinical data, and program participation records. It includes metrics on healthcare utilization, financial performance, clinical outcomes, and patient adherence to treatment regimens. Additionally, in some embodiments, this data encompasses predictive analytics on risk stratification, care gaps, and potential interventions. The data set also integrates benchmarking against normative data or best practices, thereby enabling healthcare providers and payers to measure the effectiveness of their services against established standards.

In some embodiments, pricing data, such as data generated from a proprietary pricing engine, includes, but is not limited to, extensive data sets focused on the financial aspects of healthcare services. It encapsulates information on current market rates for various medical procedures and services, pharmaceutical pricing, and the costs associated with different healthcare providers. The data comprises details on negotiated contract rates, reimbursement models, historical pricing trends, and comparative analysis across different regions or service providers. Additionally, in some embodiments, the pricing data may integrate cost forecasting, budget impact models, and scenario analyses

In some embodiments, risk and quality analytics data, such as data generated from a proprietary analytics engine, includes but is not limited to, an array of data points that enable evaluation and monitoring of the quality, efficiency, and safety of healthcare delivery that encompasses risk assessments, quality measures, patient safety indicators, and compliance with clinical guidelines. The risk and quality analytics data includes outcomes data, risk adjustment factors, and analytics related to population health management. Additionally, in some embodiments, the risk and quality analytics data includes data related to care management programs, member health assessments, and provider performance evaluations.

In some embodiments, Healthcare Effectiveness Data and Information Set (HEDIS) quality metrics data includes, but is not limited to, one or more standardized performance measures that are used to assess the quality of care and services provided by health plans. The HEDIS quality metrics data includes one or more indicators across various domains of care, including preventive health services, chronic disease management, mental health care, substance use treatment, care coordination, and the like. It includes data related to healthcare effectiveness, patient safety, timeliness of care, and patient engagement. Additionally, in some embodiments, the HEDIS quality metric data may also encompass measures of utilization and risk-adjusted health outcomes.

In some embodiments, clinical prediction analytics data includes, but is not limited to, patient demographics, historical clinical data, treatment records, real-time health monitoring data, and the like. The prediction analytics data, in some embodiments, includes data generated by one or more predictive models and algorithms that analyze patterns in the data to anticipate future health events, such as hospital readmissions, disease progression, or the likelihood of specific health conditions developing. Additionally, in some embodiments, the clinical prediction analytics data includes data indicative of risk scores, potential gaps in care, and suggested preventative measures.

In some embodiments, financial savings factors data includes, but is not limited to, data related to cost avoidance, reduction in unnecessary medical procedures, efficiencies gained through improved care coordination, and savings from formulary management in pharmacy benefits. Additionally, in some embodiments, the financial savings factors data includes data on member cost-sharing amounts, provider network contracting savings, and the impact of wellness programs on overall healthcare costs.

In some embodiments, Social Determinants of Health (SDoH) data includes, but is not limited to, data points related to non-medical factors influencing patient health outcomes. This data encompasses socio-economic status, education level, neighborhood and physical environment, employment status, social support networks, and the like. The SDoH data, in some embodiments, includes information collected through patient surveys, community health assessments, and public health databases. Additionally, in some embodiments, the SDoH data includes indicators of health disparities, access to healthcare services, and environmental risk factors.

In some embodiments, NYU Avoidable Preventable classification data includes, but is not limited to, data related to metrics that categorize healthcare events deemed either avoidable or preventable with proper and timely medical care, patient education, and other interventions. This classification data includes data elements such as emergency department visits that could be managed in primary care settings, hospital admissions for conditions preventable through outpatient services, and incidences of chronic disease complications that can be mitigated through proper management and lifestyle adjustments. Further, the SDoH data includes measures of healthcare system efficiency, patient engagement in preventive care, and effectiveness of community health initiatives.

In some embodiments, Area Deprivation Index (ADI) data includes, but is not limited to, data that ranks neighborhoods by socioeconomic status disadvantage in a region or across the nation. The ADI data includes data related to as income, education, employment, housing quality, and other socioeconomic factors which demonstrate disparities across different regions. The ADI data, in some embodiments, is arranged by region, such as by zip code.

In some embodiments, Admit, Discharge, and Transfer (ADT) data includes, but is not limited to, operational data detailing patient movement within a healthcare facility or across facilities. This data set includes timestamps and related information for patient admissions, discharges, and transfers among different departments or care settings. The ADT data, in some embodiments, is collected in real-time, facilitating immediate updates to a patient's status and location. Additionally, in some embodiments, the ADT data includes identifiers that can be used to track patient flow, manage bed occupancy, and coordinate care transitions effectively.

In some embodiments, Rural-urban Commuting Area (RUCA) data includes, but is not limited to, data that categorizes regions, such as U.S. census tracts, using measures of population density, urbanization, and daily commuting. The RUCA data, in some embodiments, provides a data relating to the rural-urban continuum, distinguishing between areas, such as metro and rural. Additionally, in some embodiments, the RUCA data includes the primary commuting flows to identify the social and economic integration of locales.

The data is ingested into the system via multiple pathways, thereby providing flexibility in the collection mechanism. Specifically, one pathway includes an Application Programming Interface (API) that establishes a secure communication channel for automated data transfer between the data collection module 122 and external data sources, thus facilitating real-time or batch-based data acquisition. Another pathway allows for manual input by authorized users via a dedicated user interface, where such input can be executed through file uploads or direct data entry into predefined fields. Additionally, data intake can be accomplished through third-party integrations, middleware, or direct database queries that serve to populate the database 125. The data collection module 122 further incorporates data validation and integrity checks to ensure the consistency and reliability of the ingested data. By offering a plurality of data intake methodologies, the data collection module 122 ensures robust and comprehensive data assimilation for downstream processing.

The data processing module 124 of the value impact platform 120 partakes in the processing and preparation of the data for further analysis by the healthcare management module 126. The data processing module 124 engages in the cleaning of the data, removal of irrelevant or redundant information, and conversion of the data into a format suitable for further processing by the healthcare management module 126. The data processing module 124 is configured to augment the initial data collection by transforming the raw, heterogeneous data into a unified, standard format, which is useful for accurate and efficient downstream processing. Specifically, the data processing module 124 executes a series of algorithms responsible for data standardization, thereby reconciling discrepancies in data types, units, or terminologies originating from disparate sources.

The data processing module 124 also integrates error-handling mechanisms to identify and rectify potential data inaccuracies or anomalies. Such mechanisms may involve rule-based checks, probabilistic data matching, or data imputation techniques, all aimed at preserving data quality and integrity. Furthermore, the data processing module 124 may incorporate parallel processing capabilities to concurrently handle multiple data streams, thereby ensuring timely and efficient data throughput. This is particularly advantageous when dealing with large-scale data sets or real-time analytics where swift data processing is desired.

The healthcare management module 126, upon receiving the prepared data from data processing module 124, applies algorithms and models, such as healthcare management model 127, to generate one or more data objects including one or more healthcare management metrics, based on the input data. The healthcare management module 126 utilizes various algorithms and employs a variety of models to accomplish its task. The healthcare management module 126 engages in the computational manipulation of the ingested data. Utilizing the healthcare management model 127 as one among a possible array of analytical frameworks, the healthcare management module 126 applies a combination of algorithmic and machine-learning methodologies to generate one or more healthcare management metrics based on the input data. Such metrics serve as quantifiable representations of various aspects of healthcare management.

In some embodiments, the healthcare management module 126 applies algorithms related to clinical opportunities methodology. This methodology integrates diverse sets of processed data, such as medical claims, financial data, and clinical histories, to produce a healthcare management metric that reflects opportunities for cost and quality optimization in healthcare delivery.

In another embodiment, the healthcare management module 126 employs machine-learning-based prediction algorithms to produce metrics that predict future healthcare events. These could include patient risk stratification or likelihood of hospital readmission, or the like. The predictive models, which are a part of the healthcare management model 127, use features extracted from the processed data, such as social determinants of health, historical medical data, area deprivation index scores, one or more other features extracted from the processed data as discussed herein, or a combination thereof.

Additionally, the healthcare management module 126 in some embodiments uses value impact modeling to generate healthcare management metrics that evaluate the resource efficiency (such as economic, staffing, or material usage implications) of distinct clinical interventions or pathways. These metrics are derived from simulations that are conducted using various models, each designed to measure the financial impact of specific healthcare decisions.

The healthcare management module 126, in some embodiments, further produces healthcare management metrics that represent aggregated patient worklists or next-best-action recommendations. These metrics are formulated through a combination of rule-based algorithms and probabilistic models, which evaluate and incorporate variables like HEDIS quality metrics and medical and pharmacy claims.

After the healthcare management module 126 has generated the one or more data objects including one or more healthcare management metrics based on the input data, a user interface generated on a user device via the user interface module 128 displays the results to the user at an appropriate time. The user interface provides an interactive and intuitive interface, enabling the user to view, modify, or confirm the generated results. The user interface also enables the user to provide feedback or additional information to improve the healthcare management process or adjust the healthcare management model 127 accordingly. The user interface module 128 is also configured to receive a user input via an interactive interface, the user input being one or more parameters.

FIG. 1C is a diagram of example components of a healthcare management module 126, according to some embodiments of the disclosure. FIG. 1C provides a more detailed view of the healthcare management module 126 and its relationship with the healthcare management model 127 within the value impact platform 120. As depicted, the healthcare management module 126 includes a healthcare management model 127. The healthcare management model 127 is configured or trained to determine appropriate healthcare management metrics, in the form of one or more data objects, related to resource utilization, care adherence, care outcomes, resource efficiency, and the like, based on various factors, such as those reflected in the health data 110. Furthermore, the healthcare management model 127 also takes into account changes to the health data and/or to the populations within the health data to increase the likelihood of an accurate response.

The healthcare management model 127, as part of the healthcare management module 126, orchestrates the creation of healthcare management metrics, such as data objects, from health data 110. This algorithm is agnostic to its underlying implementations and is designed to accommodate various types of algorithms, either individually or in combination, to achieve the desired outcomes. In some embodiments, the healthcare management metrics generated by the healthcare management model 127 pertain to predicted utilization of resources and services, projected complexity of medication regimens, identified categories associated with risks and/or severities, or other relevant aspects related to patient care and treatment planning. It should be noted that while the described implementation involves a predicative model, alternative configurations incorporate other models or approaches depending upon the specific needs and requirements of the healthcare facility and patients served. For example, the healthcare management model 127, in some embodiments, analyzes historical patterns in healthcare usage data to develop predictions about future trends. This information is then be used to optimize staffing levels, inventory management, equipment maintenance schedules, and other logistical considerations necessary for providing efficient and effective medical care. Additionally, the generated metrics assist clinicians in identifying patients who would benefit from targeted interventions or early discharge planning efforts, thereby reducing hospital stays and improving overall patient health outcomes.

In some embodiments, the value impact platform 120 is configured to support contract ingestion and standardization. The data collection module 122 is configured to receive contract terms among other types of healthcare-related data. Upon collection, these contract terms are forwarded to the data processing module 124. The data processing module transforms the heterogeneous contract data into a unified, structured format that is suitable for subsequent processing by the healthcare management module 126 and storage within the database 125.

The data processing module 124 employs algorithms designed specifically for contract standardization. These algorithms reconcile variances in contract terminologies, units, and conditions, thereby eliminating inconsistencies that could potentially impact the quality of the generated healthcare management metrics. This standardization process results in the normalization or standardization of contracts from disparate sources that can be accurately compared, analyzed, and integrated within the overarching healthcare management framework enabled by the value impact platform 120.

In addition to terminology reconciliation, the data processing module 124 performs the task of structuring the ingested contract data. This involves breaking down complex contract clauses into constituent elements, which are then mapped to predefined fields within the database 125. By doing so, the data processing module 124 ensures that the contract data is organized in a manner conducive to efficient query execution and data retrieval. Following the completion of the contract ingestion and standardization process, the standardized contract data is stored in the database 125 and is made accessible to the healthcare management module 126 for subsequent analytical operations.

In some embodiments, the data processing module 124 is configured to combine two or more contracts for the purpose of generating healthcare management metrics. The platform identifies contracts with terms sufficiently similar to warrant amalgamation into a single data object. Subsequently, these unified contract data objects are stored in the database 125 and are rendered accessible to the healthcare management module 126 for further analytical activities. The data processing module 124 incorporates rules-based mechanisms or utilizes one or more models or algorithms to establish the suitability of combining specific contracts. In a rules-based approach, pre-defined combination rules are set by one or more users of the system. These rules specify criteria that contract terms must meet to be considered similar, such as identical service categories, payment models, geographical locations, or the like. In some embodiments, the data processing module 124 employs computational models or algorithms to assess the suitability of contracts for combination. These algorithms analyze attributes such as contract duration, parties involved, and other contractual elements, and apply statistical or machine-learning techniques to make determinations on whether contracts can be combined together.

Once contracts are combined into single data objects, the user, through the user interface module 128, is enabled to select these combinations for analysis. The healthcare management module 126 then generates one or more healthcare management metrics or reports based on the combined contract data objects. The system is further designed to allow the user to modify the selection of combined contracts. Upon such re-selection, the healthcare management module 126 automatically re-generates the healthcare management metrics or reports to reflect the updated corpus of selected contracts.

In some embodiments, the value impact platform 120 generates one or more performance reports for the individual or combined contracts. This performance reporting is formulated based on a combination of input data and the standardized or amalgamated contract data objects stored in the database 125.

In some embodiments, the healthcare management module 126 employs algorithms related to financial performance reports. These algorithms integrate the standardized contract data with other forms of healthcare data, such as medical and pharmacy claims, HEDIS quality metrics, clinical prediction analytics, or the like, to yield a performance report that assesses the financial implications of the individual or combined contracts. The report covers aspects such as cost-efficiency, quality of care, and adherence to contract terms, among other criteria.

In another embodiment, the healthcare management module 126 uses the clinical opportunity identification methodology to generate performance reports. This methodology combines the contract data, whether individual or combined, with clinical histories, social determinants of health, or other relevant healthcare data to identify opportunities for clinical improvements and cost savings. The resulting performance report would provide a granular analysis of the efficacy and efficiency of healthcare service delivery as stipulated by the contract terms.

For contracts that have been combined, the healthcare management module 126 is configured to generate a unified performance report that represents the aggregated impact of the bundled contracts. This unified report would comprise metrics such as cumulative cost savings, overall quality improvement, and combined compliance rates, synthesized from the individual contracts included in the combination.

Further, in some embodiments, the system enables the user, through the user interface module 128, to interact with the generated reports. Users can select different combinations of contracts, prompting the healthcare management module 126 to re-calculate and re-generate performance reports based on the newly selected combinations. This adaptability ensures that users obtain tailored insights that cater to different analytical needs.

In some embodiments, the healthcare management module 126 is configured to perform tasks related to clinical and quality modeling. The module receives input data and standardized or combined contract data from the database 125 and applies a series of algorithms and models for the generation of clinical and quality metrics. These metrics pertain to the assessment of healthcare services, patient outcomes, and compliance with established healthcare standards. The healthcare management module 126 incorporates specific algorithms designated for evaluating quality metrics such as HEDIS scores, patient satisfaction rates, and clinical effectiveness measures. These algorithms integrate with the contract data to discern how specific contractual terms and conditions influence quality outcomes. For example, an algorithm assesses how a payment model specified in a contract impacts the healthcare provider's adherence to HEDIS standards. Similarly, the module includes clinical modeling capabilities that employ advanced algorithms or machine-learning models. These clinical models incorporate multiple variables from the input data, including but not limited to medical and pharmacy claims, member eligibility, and social determinants of health, to produce actionable insights. For instance, the module could utilize an algorithm that integrates patient medical histories and contract-specific guidelines on pharmaceutical usage to determine optimal drug regimens for individual patients. Moreover, the clinical and quality metrics generated can be included as part of broader performance reports. These reports are displayed through the user interface module 128, which allows users to interact with and interpret the metrics, thereby enabling more informed healthcare management decisions.

In instances where combined contracts are used, the healthcare management module 126 is further configured to generate clinical and quality models that reflect the aggregate effect of these combined contracts. For instance, a unified quality model might be generated that blends the quality metrics from multiple contracts to offer a holistic view of healthcare service quality across an entire healthcare network.

In some embodiments, the healthcare management module 126 incorporates functionalities designed for dynamic scenario modeling. Specifically, the module enables the modeling of scenarios that simulate the impact of various improvement opportunities on performance metrics, particularly with respect to financial, clinical, and quality dimensions. This capability allows users to forecast the outcomes of potential actions or interventions within the healthcare system. For instance, the dynamic scenario modeling employs a modeler which is configured to capture the top n common payer scenarios. This modeler assimilates information from diverse data sources such as financial models, clinical histories, and quality metrics, all of which are stored in the database 125. The modeler then utilizes these data points in conjunction with the contract data, whether individual or combined, to generate a set of scenario options.

Understanding that medical groups often operate under resource constraints, the dynamic scenario modeler allows the user to selectively focus efforts on one or two targeted metrics with the objective of optimizing performance against contractual targets. Users interact with this feature via the user interface module 128, where they can specify the level of resource allocation they wish to devote to particular opportunities for improvement. For example, in some embodiments, a user might elect to focus on optimizing HEDIS quality metrics. The dynamic scenario modeler would then simulate the impact of such an optimization on financial performance, considering parameters such as reimbursement rates stipulated in the contract or contracts. Simultaneously, the dynamic scenario modeler would also forecast the implications on clinical performance metrics, such as patient health outcomes or admission rates. By way of another example, in some embodiments, the user could decide to emphasize efforts on cost-saving measures in pharmaceutical spending. Here, the dynamic scenario modeler generates a scenario illustrating how such an effort would affect not just financial metrics like overall spending, but also quality metrics like patient satisfaction and clinical efficacy. In cases involving combined contracts, the dynamic scenario modeler is further configured to aggregate the impacts across the multiple contracts, providing a consolidated view of how resource allocation in selected areas would influence performance metrics at a holistic level.

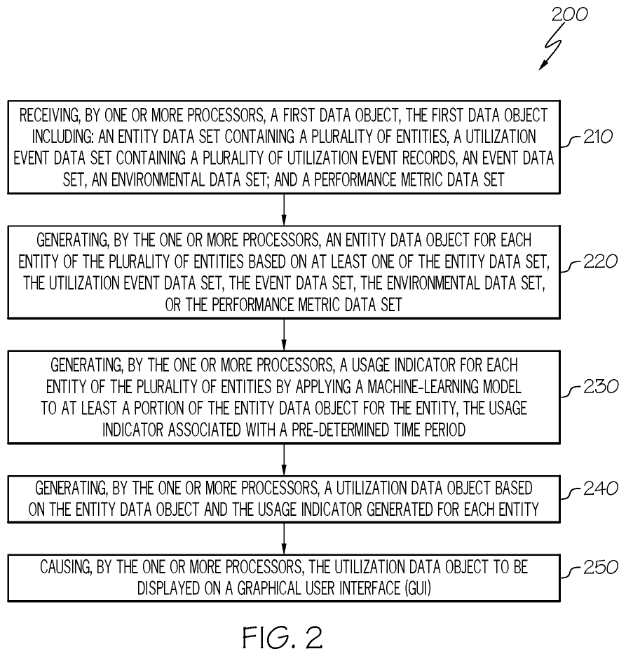

FIG. 2 is a flowchart showing a method 200 for determining unnecessary internal system utilization. In step 210, the value impact modeler 120 receives a first data object. The first data object may comprise one data object or a plurality of data objects, that includes a collection of data sets. In some embodiments, the first data object includes an entity data set containing a plurality of entities. The entity data set encompasses data about one or more members, with each member potentially being associated with one or more providers, such as a healthcare provider.

In some embodiments, the first data object includes a utilization event data set. The utilization event data set includes a plurality of utilization event records, which include information about one or more medical claims and/or pharmacy claims associated with one or more entities. The value impact platform 120 utilizes the information within the utilization event data set to understand internal system resource utilization related to the claims, along with utilizing the data within the utilization event data set as an input to one or more additional models and/or as the basis for generating one or more additional metrics.

The first data object also comprises an event data set. This event data set is versatile, allowing for the inclusion of one or more data arrays, such as an episode treatment groupers (ETGs) array, a service categories array, an Agency for Healthcare Research and Quality (AHRQ) groupers on medical conditions array, or the like. In some embodiments, ETGs are a classification system used in healthcare to group clinically similar medical events. The ETGs array includes information which is utilized and applied to medical events, such as medical claims data, to aggregate individual, patient-specific medical claims and encounter data into clinically meaningful and discrete units, known as “episodes of care.” Each episode represents a distinct phase of a patient's medical treatment, from initial diagnosis through the course of treatment for a particular condition. In some embodiments, the groupers are associated with and/or utilized to identify one or more chronic conditions, such as asthma, CHF, COPD, diabetes, hypertension, or the like.

The service categories array is a systematic classification framework to categorize various medical services and procedures. This array breaks down the multitude of healthcare services into more manageable and distinct categories, making it easier to analyze and understand healthcare operations. Within the service categories array, individual services or procedures are grouped based on their nature, purpose, or the medical specialty they pertain to. For instance, services related to cardiology, neurology, orthopedics, or radiology may each form distinct categories. Moreover, the array could further classify services based on factors like the type of care (e.g., preventative, diagnostic, therapeutic), setting (e.g., inpatient, outpatient), or the severity and complexity of the case. By employing such a categorization system, healthcare providers, payers, and analysts can achieve a clearer perspective on the distribution, utilization, and costs associated with different types of medical services.

In some embodiments, the event data set includes an AHRQ groupers data array. The AHRQ groupers data array encompasses a collection of clinically coherent groupings that categorize medical conditions using a standardized methodology. Each grouping in the AHRQ groupers data array is designed to aggregate medical claims into specific categories, thereby facilitating a more structured analysis of the healthcare data. The array includes specific codes or identifiers that correspond to different medical conditions, treatments, or procedures. These identifiers assist in the organization and interpretation of large sets of medical data, enabling more effective utilization analysis. Furthermore, the AHRQ groupers data array, in some embodiments, intakes new data and is dynamically updated to reflect changes in medical practice, new treatments, or emerging health conditions. In conjunction with other data sets, such as the entity data set, utilization event data set, and environmental data set, the AHRQ groupers data array aids in providing a comprehensive view of internal system utilization patterns, thus allowing the value impact platform 120 to derive insightful metrics and predictions regarding future healthcare needs and potential interventions.

In some embodiments, the first data object includes an environmental data set. The environmental data sets include data sets and arrays which relate to one or more environmental aspects associated with healthcare. For example, the environmental data set includes, in some embodiments, an Area Deprivation Index (ADI) data set. The ADI data set provides indicators pertaining to geographical regions reflecting socio-economic challenges based on factors such as income and education levels. Specifically, the ADI data set contains indicators that rank regions based on their deprivation scores. These scores are derived from comprehensive evaluations of various factors, including but not limited to, household income, employment rates, access to education, and other socio-economic determinants. Such data assists in recognizing regions where residents might be at a higher risk for health disparities due to socio-economic challenges. When integrated with other data, such as the entity data set or the event data set, the ADI data set offers a deeper context, potentially highlighting correlations between geographical deprivation and health outcomes. This, in turn, aids the value impact platform 120 in generating more holistic and accurate prediction indicators.

The environmental data set also includes, in some embodiments, a social determinants of health (SDoH) data set. In some embodiments, the SDoH (Social Determinants of Health) data set includes a broad spectrum of non-medical factors that influence health outcomes for one or more members within the entity data set. This encompasses socio-economic data such as income levels, educational attainment, employment status, and housing stability. It also includes data about a patient's social and community context, including social support networks, community engagement, and potential exposure to violence or crime. Environmental factors, such as access to clean water and safe housing, proximity to parks or recreational areas, and potential exposure to environmental toxins, are, in some embodiments, also included in the data set.

In some embodiments, a rural/urban data set (RUCA) comprising rural-urban commuting area codes is included in the environmental data sets. The RUCA data set delineates geographical regions based on how urban or rural the geographical region is and the nature of work-related commutes. Specifically, these codes categorize regions into urban, rural, transitional areas, and the like, offering insights into the dynamics of population density, infrastructure, and accessibility to health and other services. The RUCA data set can be utilized to determine the relative availability and reach of healthcare facilities, transport systems, and local amenities within these designated areas.

Furthermore, when interfaced with the entity data set or the event data set, the RUCA data set serves to contextualize health data 110 within the broader framework of urban-rural divides. This aids in highlighting disparities in healthcare access, service quality, and health outcomes across different community types. For instance, members from rural areas may have different healthcare needs or face different challenges than those in urban locales, such as longer travel times to health facilities or limited access to specialist care.

The first data object is also supplemented by a plurality of data sets associated with one or more performance metrics (e.g., a performance metric data set). These performance metrics include additional health data, consisting of a provider data set which relates to one or more providers, insurers, hospital service locations, or the like. The plurality of data sets associated with one or more performance metrics, in some embodiments, also contains indicators and/or data related to access to health care, including proximity to healthcare facilities, transportation options, and health insurance status. Behavioral data, including dietary habits, physical activity levels, tobacco and alcohol use, and other substance use or misuse, are, in some embodiments, present. In some embodiments, the performance metrics can also include data that is not covered by the entity data set, utilization event data set, event data set, and environmental data set, and that were otherwise discussed elsewhere in the present disclosure.

In some embodiments, at step 220, the method includes generating, by the one or more processors, an entity data object for each entity of the plurality of entities. In some embodiments, the entity data object is generated based on at least one or more of the entity data set, the utilization event data set, the event data set, the environmental data set, or the performance metric data set.

The entity data object serves as a comprehensive record for each entity (e.g., each patient), integrating multiple data points and parameters from various data sets to provide a holistic view of the entity's health and utilization profile. In some embodiments, to generate the entity data object, the value impact platform 120 performs one or more data modeling steps. These steps involve algorithms or computational models that transform raw data into structured and meaningful information pertaining to each entity.

In some embodiments, the data modeling includes generating one or more entity state data points. The entity state data point represents the condition or status of the entity at specific moments in time or during the occurrence of clinically significant events, such as an emergency department visit or the occurrence of an inpatient utilization. This provides a snapshot of the entity's health or utilization state, aiding in the predictive analysis of future healthcare needs or patterns.

Additionally, in some embodiments, the data modeling process involves creating a drug count for the entity based on pharmacy claims. This count provides insights into the entity's medication usage, adherence, and potential drug interactions. In parallel, the system applies episode treatment groupers (ETGs) from the event data set to the medical claims, flagging entities that exhibit specific chronic conditions (e.g., entities that exhibit ambulatory care sensitive condition (ACSC)). This step aids in categorizing and understanding the entity's health challenges and associated healthcare utilization patterns.

In some embodiments, the data modeling also encompasses the application of service categories to medical claims, helping to classify the types and nature of medical services availed by the entity. The generation of the entity data object, in turn, amalgamates various determinants of health, such as ADI, RUCA, ETGs, service categories, AHRQ groupers, and data derived from the modeling steps, culminating in a singular, comprehensive record for each entity. Such a record facilitates the systematic analysis of health trends, prediction of future utilization patterns, and the identification of intervention opportunities, thereby optimizing healthcare management through the healthcare management module 126 or the like. In some embodiments, the entity data object is created for ACSC-chronic entities that were identified during or as a result of the data modeling process described above.

In some embodiments, at step 230, the value impact platform 120 generates a usage indicator for each entity of the plurality of entities (or a portion of the plurality of entities that are ACSC chronic, as identified during or as a result of the data modeling process described above). In some embodiments, the usage indicator is generated based on at least a portion of the first data objects. For example, the usage indicator is generated based on one or more of the entity data set, the utilization event data set, the event data set, the environmental data set, or the performance metric data set. In some embodiments, the usage indicator is generated based on at least a portion of the entity data object generated for the entity. The usage indicator is, in some embodiments, associated with a pre-determined time period. For example, in some embodiments the usage indicator is indicative of an anticipated use of one or more internal system resources within an upcoming 12-month time frame.

The value impact platform 120 utilizes the usage indicator in predicting healthcare utilization patterns for each entity. It encapsulates a combination of historical data, current health status, environmental factors, and other relevant metrics to formulate a predictive measure of internal system resource utilization for a given entity within the specified time frame.

In some embodiments, generating the usage indicator involves applying one or more machine-learning models to at least a portion of the first data object (e.g., one or more of the entity data set, the utilization event data set, the event data set, the environmental data set, or the performance metric data set). In some embodiments, generating the usage indicator involves applying one or more machine-learning models to at least a portion of the entity data object generated for each entity. These models, which are trained, adjusted, modified, and/or taught, to identify association, patterns and correlations from vast amounts of data, and harnessed by the value impact platform 120 to make informed predictions about the likelihood of an entity using specific healthcare resources (e.g., likelihood of an entity utilizing internal system resources). Depending on the focus of the system and/or a desired output from the user, the machine-learning model may be trained specifically for predicting internal system resource utilization such as, e.g., emergency room (ER) utilization (e.g., emergency resource utilization), inpatient service (IP) utilization (e.g., internal services utilization), or a combination of both. In some embodiments, the internal system utilization includes at least one of emergency resource utilization or internal services utilization, and the machine-learning model is trained based at least in part on i) training data set associated with a plurality of sample entities, the training data set including at least one of a sample entity data set, a sample utilization event data set, a sample event data set, a sample environmental data set, or a sample performance metric data set, and ii) respective probabilities of internal system utilization associated with the plurality of sample entities.

In some embodiments, a logistic regression model is used as the machine-learning model. In some cases, a model ensemble, such as a Mixture of Experts (MoE) model, might be employed to leverage the strengths of individual models and provide more accurate predictions across a range of scenarios.

The data fed into these models is multifaceted and derived from various sources. For ER utilization predictions, the model in some embodiments incorporates historical data on past ER visits, current medications, recent diagnostic tests, and any known chronic conditions. Data points like frequency of previous ER visits, duration since the last visit, and reasons for prior visits can be utilized as inputs to the model. For inpatient service utilization predictions, the model considers past hospitalization records, ongoing treatments, and other significant health events such as surgeries or specialized procedures. Furthermore, environmental factors from the environmental data set, such as the entity's geographical location, proximity to healthcare facilities, and socio-economic indicators, are integrated into the model. These factors influence health-seeking behaviors and access to healthcare resources. Additionally, insights from the performance metric data set, which encapsulates health metrics and outcomes, are utilized to enhance the predictive accuracy of the model. In some embodiments, the model is applied to the data within one or more entity data object.

In some embodiments, the machine-learning model not only predicts the likelihood of utilization but also the potential intensity or duration of the utilization. For instance, whether an ER visit might escalate to an inpatient admission or if a patient is likely to have prolonged hospital stays.

The application of the machine learning model results in a usage indicator, which provides a data-driven flag or signal that quantifies the probability of a particular entity (member) engaging in preventable resource utilization. For instance, based on the comprehensive entity data record, the model could forecast the likelihood of an entity visiting an emergency department or requiring inpatient care within the upcoming year.

Further, in some embodiments, the usage indicator is associated with a risk score. This risk score quantifies the probability of internal system utilization by the respective entity during the predetermined period (e.g., in the next 6 months, 12 months, 18 months, 24 months, etc.). Such risk scores can be specialized, with some scores focusing on emergency department utilization, others on inpatient service utilization, and yet others combining various resource utilizations for a comprehensive risk assessment.

In some embodiments, the risk score serves as a quantifiable metric that gauges the likelihood of a particular resource utilization by an entity in a specified upcoming time period. This metric, generated by machine-learning models, is rooted in the intricate analysis of various data sets, ranging from historical utilization data to current health metrics and environmental factors.

The association with the risk score is structured to capture the granularity of healthcare utilization patterns. In some embodiments, individual risk scores are generated for each type of resource utilization, such as emergency room utilization or inpatient utilization. This allows stakeholders to have a differentiated view of the potential healthcare requirements of an entity. For instance, a high ER risk score might suggest frequent, short-term medical emergencies, whereas a high IP risk score could imply prolonged hospital stays or significant medical interventions. In some embodiments, a comprehensive risk score is formulated which amalgamates the likelihoods of various resource utilizations into a single score. This holistic approach provides a consolidated view of an entity's overall anticipated strain on the healthcare system.

The generation of these risk scores by the value impact platform 120 is grounded in the capabilities of one or more machine-learning models. In some embodiments, a model is specifically trained to determine associations between entity data, usage indicators for emergency room utilization and/or inpatient utilizations, and the respective risk scores. The depth and breadth of the input data, which includes one or more data from the first data set and any additional data generated during the method, empower the model to generate accurate and actionable risk scores.

While a single model can offer significant insights, in some embodiments, multiple machine-learning models are employed to enhance the accuracy and specificity of the risk predictions. Each model in such a multi-model approach can be fine-tuned to identify associations for specific types of resource utilization. For example, one model might be optimized for predicting ER utilization based on factors like past ER visits and known chronic conditions. Simultaneously, another model might focus on predicting inpatient services utilization, factoring in parameters like ongoing treatments and past hospitalization records.

In some embodiments, at step 240, the value impact platform 120 generates a utilization data object based on the previously derived entity data object and the generated usage indicator for each entity (e.g., for each entity that is ACSC chronic). This utilization data object serves as a centralized repository of predicted internal system resource utilizations for each entity within a specified future period, such as the next 12 months.

The generation of this utilization data object involves using one or more classification codes extracted from the performance metric data set. These classification codes are designed to identify specific preventable utilization events, offering a clear insight into possible healthcare encounters that could potentially be avoided with timely interventions. By categorizing these predicted utilizations, the system empowers healthcare providers to prioritize resources and interventions for high-risk entities.

In some embodiments, the utilization data object presents a list of entities predicted to have emergency room or inpatient utilizations within the upcoming time frame. The precision of these predictions can be enhanced by leveraging specific data sets like AHRQ, NYU, or other classification data sets. These data sets assist in predicting the nature of the expected claim, based on historical data of the entity, and further discern if such a claim or healthcare encounter is avoidable.

If the value impact platform 120 indicates a claim as avoidable based on one or more classifications as applied to entity data, additional details are procured to inform the prediction. In cases of anticipated emergency room utilization, in one example, procedure codes are used to determine the severity of the potential visit. Furthermore, geographical data, such as zip codes, is used to estimate ER/IP costs. An intervention data set is also leveraged by the value impact platform 120 to ascertain the costs associated with potential preventive measures.

Building on this data, to the value impact platform 120 generate a resource efficiency metric for each entity. This metric, derived from the utilization data object and/or the associated usage indicator, offers a quantifiable measure of the expected efficiency gains from preventive actions. The resource efficiency metric is also based on the identified preventable utilization event(s) and/or the determined intervention action(s). For example, the efficiency metric is rooted in the balance between the identified preventable utilization event and the potential intervention action. As explained above, the intervention action(s) can be determined based on the utilization data object for each entity, wherein the intervention action is targeted to mitigate a likelihood of the respective entity's preventable utilization event(s).

By comparing this anticipated resource usage against historical costs, visit data for the entity, and broader system-wide data, the value impact platform 120 determines a resource offset for each planned intervention. In essence, this provides a projection of the potential resource savings or optimization if a specific intervention is applied to an entity. This resource offset, or anticipated savings, is then incorporated into the utilization data object, serving as an indicator for cost-effective healthcare management and intervention prioritization.

In some embodiments, the value impact modeler 120 identifies one or more savings opportunities. The savings opportunity is, in some embodiments, related to emergency room utilization. In some embodiments, the value impact modeler 120 identifies one or more savings opportunities related to emergency room utilization. The modeler employs classifications from the Agency for Healthcare Research and Quality (AHRQ) data and New York University (NYU) data based on diagnosis codes to ascertain which claims could potentially be prevented. Additionally, the modeler utilizes procedure codes to categorize ER severity levels, which further refines the identification of preventable claims. The value impact modeler 120 further uses provider zip codes to determine the costs associated with emergency room, as well as the costs of interventions, by referencing the one or more proprietary price data set, such as a Comprehensive Price Index (CompPricer) database. An estimated savings per member is calculated by taking the difference between the product of past ER costs and the number of past ER visits, and the costs associated with interventions. This estimated savings reflects the potential reduction in costs achieved by preventing unnecessary ER visits.

In some embodiments, the value impact modeler 120 identifies one or more savings opportunities related to inpatient services utilization. The modeler utilizes classifications from the Agency for Healthcare Research and Quality's (AHRQ) data set, which in some embodiments includes a Prevention Quality Indicators (PQI) data set. The PQI data set includes indicators such as admissions for chronic obstructive pulmonary disease or asthma, diabetes complications, hypertension, heart failure, dehydration, bacterial pneumonia, and urinary tract infections. These classifications aid in distinguishing which inpatient (IP) admissions could have been prevented. Further, the value impact modeler 120 employs provider zip codes to ascertain inpatient costs and potential intervention costs using one or more proprietary price data sets, such as a Comprehensive Price Index (CompPricer) database. The estimated savings per member are computed by taking the difference between the product of past inpatient costs and past inpatient visits, and the costs associated with interventions.

By way of example, consider a scenario in a given healthcare system where a specific group of patients, identified as the “at-risk” population, is vulnerable to severe complications from the influenza virus. This at-risk group may vary each year based on a combination of environmental factors, such as the prevalence and strain of the flu virus in the community, and the chronic conditions identified within the population. For instance, one year, the at-risk group may be predominantly comprised of elderly individuals, while the next year, it could include more patients with respiratory conditions due to increased pollution levels. The at-risk population, or the population which benefits from intervention, is determined by the value impact platform 120 applying one or more entity data objects to one or more machine learning model to identify, based on entity data and environmental data, the risk for each member within the population. The at-risk population is a population for which the generated utilization indicator shows a likelihood of utilization of one or more resources, such as emergency room utilization or an inpatient stay during the next 12 months.

Continuing the example, without any interventions, the expected hospitalization cost due to flu-related complications for this at-risk population over a flu season is $2,500,000. These costs arise from emergency room visits, inpatient hospital stays, and associated medical treatments. The costs are derived by the value impact platform 120 applying historical costs to the predicting utilizations.

Continuing the example, an intervention, in the form of a targeted flu shot campaign tailored to the at-risk population, is proposed, either by the value impact platform 120 itself or by a user leveraging one or more features of the value impact platform 120. This campaign includes not only the cost of the vaccine but also educational sessions, outreach programs, and follow-up consultations. With this intervention in place, the expected hospitalization cost due to flu-related complications is projected to reduce to $1,200,000. The total cost to implement the flu shot campaign, inclusive of all its components, is $300,000. Thus, the total resource utilization offset for the at-risk population is $2,500,000−($1,200,000+$300,000)=$1,000,000, while also providing better health outcomes for the at-risk population. This resource utilization offset is, in some embodiments, comprehensive of the at-risk population, while in some embodiments the offset is applied at an entity data object level, so that the intervention is identified on a per-member basis.

In some embodiments, the value impact platform 120 applies the calculation of resource utilization to each entity individually. In doing so, the value impact platform 120 enables modeling of one or more scenarios or end goals of the user. Each entity, or patient, has unique characteristics, medical histories, and risk factors. By computing the resource utilization offset at an individual level, the value impact platform 120 captures the nuances and specific needs of each patient. This ensures that the interventions proposed are not just generalized recommendations but are tailored to provide maximum benefit to each individual.

For example, this individual entity allocation of resource utilization enables assessment of the cost and method of the recommended intervention for each entity. For instance, one patient might benefit most from regular check-ups, while another might require a specific medication regimen. By integrating these individualized intervention costs and methods, the system provides a comprehensive view of both the anticipated savings and the investment required for each entity. By presenting individualized data, the platform empowers healthcare providers or administrators to make informed decisions based on various strategic objectives and supports scenario modeling by the value impact platform 120. For example, if a user indicates a goal is to maximize resource efficiency, the user can target entities where the difference between the resource utilization offset and the intervention cost is the greatest, ensuring the lowest possible costs, and the value impact modeling can perform one or more optimization step to generate worklists and scenarios which reflect interventions that target this outcome. By way of another example, if the user-defined objective is to enhance health outcomes for the largest portion of patients, the platform outputs user worklists, such as in the form of a utilization data object, which includes members with interventions that cater to larger sub-groups within the at-risk population, ensuring broader health improvements, even if the overall resource utilization reduction is not maximized. By way of another example, in cases where healthcare providers have specific contract terms or performance metrics to meet, the individualized data supports the generation of a utilization data object that offers entities and corresponding interventions and/or offsets that directly align with these contractual obligations.

The value impact platform 120, in utilizing one or more machine-learning models within the healthcare management model 127, continuously refines its predictions and recommendations based on incoming data. As interventions are implemented and outcomes observed, the platform 120 learns and adjusts, ensuring that future recommendations are even more accurate and effective.

In some embodiments, at step 250, the value impact platform 120 causes the utilization data object, or a portion of the utilization data object, to be displayed on a graphical user interface (GUI), a component of the user interface module 128 of the value impact platform 120. This GUI is designed not only to present the data in an interactive and user-friendly manner but also to offer features that enhance the user's ability to interpret and make decisions. The GUI provides interactive features such as a sortable charts, filters, categories, and modeling tools, and users are enabled to interact with specific data points, customize their dashboards based on varying interests, and delve deeper into the data for granular details. The interface is integrated within one or more other systems within the network environment 100, ensuring cross-referencing capabilities that enrich the presented insights. Additionally, the GUI, in certain embodiments, incorporates a notification system that proactively alerts users based on predetermined criteria, and provides functionalities for data export and detailed reporting. Such capabilities ensure that the users have a holistic view of anticipated resource utilizations and interventions.

In some embodiments, the value impact platform 120 incorporates a scenario modeling function, enabling users to simulate various scenarios and predict potential outcomes based on the data at hand. The scenario modeling process and results can be presented to the user visually through the GUI. This scenario modeling function allows healthcare providers and administrators to input different variables or modify existing parameters, and then observe the anticipated effects on resource utilization, intervention efficacy, or other relevant metrics. For instance, a user could model the outcome of a new intervention strategy on a specific patient subgroup, or predict resource utilization shifts in response to environmental changes. This scenario-based approach facilitates proactive planning, as stakeholders can test hypotheses, anticipate challenges, and strategize interventions in a virtual environment before actual implementation, ensuring informed and data-driven decision-making processes.

In some embodiments, one or more cluster data objects is generated. The cluster data objects are generated using one or more clustering algorithm to group the entity data objects into clusters based on common features. Each resultant cluster data objects include members that are unique compared to members of other cluster objects, allowing for interventions and care pathways to be broadly applied to multiple members by applying them to the cluster data object as a whole.

In some embodiments, one or more intervention data objects and/or arrays are generated. The intervention data object and/or array, in some embodiments, includes one or more cluster data objects and/or the members associated with the one or more cluster data objects. In the intervention data object, for each member, one or more interventions are assigned to the member. The intervention, in some embodiments, is associated with one or more alternative paths of care, which are each associated with a particular resource utilization. The value impact platform 120 determines and assigns the intervention utilizing one or more machine-learning models and/or algorithms to output an intervention that results in the most efficient resource utilization, such as by suggesting an intervention by diverting the member to an alternative care pathway based on one or more member data, the likelihood of success of the intervention, the expected resource utilization (including cost) of the intervention, and the overall reduction in resource utilization of the alternative care pathway.

Interventions are of varied types and include but are not limited to medication management, virtual nurse consultations, in-home support services, and mental health assessments. These interventions are not limited to re-admission issues and encompass a range of healthcare needs. The interventions are applied either at a member-level or at a cluster data object level. When applied at a member-level, each member receives a personalized recommended interventions based on their medical history, risk factors, and other variables such as geographic location or distance to hospital. The intervention is applied as a flag to the entity data object. When applied at a cluster data object level, all members of the particular cluster receive a common set of interventions optimized for that cluster's average or median characteristics.

The generation of interventions also incorporates an efficiency metric that accounts for the effectiveness of the interventions in reducing unnecessary resource utilizations, such as readmissions. This efficiency metric is quantified in terms of reduction in readmissions, and is often balanced against the cost of the intervention to ensure that the overall healthcare system achieves cost savings.

In some embodiments, the success of one or more interventions is tracked by the value impact platform 120. Tracking involves the monitoring and recording of key performance indicators such as utilization rate, patient satisfaction, and overall healthcare cost reduction. The collected data is subsequently used to refine the healthcare management model 127 for future scenario modeling predictions. Specifically, the realized success rates of the interventions are incorporated into the model's underlying algorithms, enabling the model to adapt and improve its accuracy in generating subsequent interventions. The ongoing integration of real-world performance data thus contributes to the continual calibration of the healthcare management model 127, thereby facilitating more precise and efficient allocation of healthcare resources and better targeting of alternative care pathways.

In some embodiments, the value impact platform 120 employs a scenario modeling technique to determine one or more possible effects of a determined intervention action on internal system utilization or intervention efficacy. The scenario modeling technique generates one or more scenario model data objects. The scenario model data object is structured to encapsulate distinct recommended focus areas, for instance, specified interventions that propel the overall member population toward particular population states. These population states are selected for their alignment with defined objectives that are stored within the scenario model data object and/or within value impact platform 120. The objectives include, but are not limited to, precise metrics such as the optimization of resource allocation, quantifiable reduction in readmission rates, measurable changes in patient health outcomes, and minimization of healthcare-related expenditures.

The generation of the scenario model data objects is performed by the value impact platform 120, which operates through data processing module 124 and the healthcare management module 126. By receiving and analyzing data from health data repository 110 and additional data sets, the scenario modeling system assesses and projects the potential ramifications of distinct interventions or modifications within environment 100.

In some embodiments, scenario modeling is executed by assigning one or more weight values to one or more metrics or outcomes associated with one or more of the data sets, to generate an optimized strategy for the healthcare system. These metrics or outcomes include, in some embodiments, a first metric such as the rate of resource utilization, a second metric such as patient readmission rates, and further metrics pertaining to patient care outcomes and cost efficiency as described herein. Each metric is attributed a specific weight that reflects its relative importance or anticipated influence on the system's overarching aims. The assignment of these weights may be initially established based on empirical healthcare data, benchmarks prevalent within the healthcare industry, or the expertise of healthcare practitioners or system administrators. Furthermore, the scenario modeling system is configured to recalibrate these weights automatically in response to shifts in population health trends or modifications in healthcare delivery contracts, such as by applying a goal-seeking algorithm and iteratively modeling varying intervention scenarios. Alternatively, the system allows for manual adjustment of these weight values by authorized users, thereby providing a dual mechanism for dynamic weight adjustment.

The weighting of metrics or outcomes allows the scenario modeling system to balance multiple considerations, such as clinical effectiveness, cost-efficiency, patient satisfaction, and regulatory compliance. For example, if the healthcare system aims to reduce unnecessary readmissions visits while maintaining a high level of patient satisfaction, the scenario modeling system can adjust the weights assigned to these outcomes to find a suitable balance of utilization reduction and alternative care pathway adoption, which in some embodiments would signify patient satisfaction with their medical care.

The user interface module 128 provides a comprehensive visualization of the scenario model data object. It allows users, such as healthcare professionals or administrators, to interact with the data, modify parameters or assumptions, and view updated projections in real-time. This interaction enables the identification of key strategies that can drive desired outcomes and optimize the healthcare system's overall performance.

Once the weights are assigned, the scenario modeling system utilizes the healthcare management model 127, which encompasses various algorithms or machine-learning models, to analyze the data and generate predictions. The system considers the relationships between different variables, the potential impact of interventions, and the feasibility of achieving desired outcomes based on the current state of the healthcare system.

Additionally, the scenario modeling system enables users to simulate various scenarios by adjusting the weights of metrics or outcomes, altering assumptions, or modifying input data. This flexibility allows for a thorough exploration of different strategies and their potential outcomes, helping decision-makers to make informed choices that align with the healthcare system's objectives. Furthermore, the scenario modeling system incorporates feedback loops for continuous improvement. As real-world data is collected and analyzed, the system refines its models and adjusts the weights of metrics or outcomes to reflect the most current and accurate information. Furthermore, the scenario modeling system can consider external factors, such as changing regulatory requirements, socio-economic conditions, or advancements in medical technology. This ensures that the system remains adaptive and forward-looking, aligning with the evolving needs of the network environment 100 and the members.

In some embodiments, the value impact platform 120 performs model monitoring. Model monitoring includes assessment one or more model performance metrics and detecting drift in the change in the statistical properties of the data that was used to train the data. In some embodiments, the drift is associated with the interventions' impact on the population, as the interventions prove successful the resulting member population metrics will, in some embodiments, drift from the metrics of the starting population. This drift, in some embodiments, is detected as new health data is populated into the system. The value impact platform 120 tracks initial parameters and/or metrics associated with the member data object and identifies changes and/or differences in those parameters over time as new data is populated into the system.

In some embodiments, the value impact platform 120 includes one or more correction mechanisms in response to the detected drift, aiming to adjust and optimize the model for altered data patterns and distributions. These correction mechanisms involve adaptive algorithms that modify model parameters, weight adjustments, or feature recalibration, ensuring that the model remains aligned with the evolving nature of the input data. In certain embodiments, the correction mechanisms employ techniques such as reinforcement learning, transfer learning, or online learning to swiftly adapt to the changing data landscape. Furthermore, these mechanisms might trigger model retraining processes, wherein new data is utilized to update the model, thereby enhancing its predictive accuracy and reliability. In other embodiments, when significant drift is detected, the correction mechanisms might recommend a comprehensive overhaul of the model, encompassing the incorporation of novel features, adjustment of hyperparameters, or even the selection of an alternative modeling approach, thereby maintaining the model's efficacy in dynamically changing environments.

In some embodiments, the value impact platform 120 includes a fairness monitoring to ensure equitable model performance across diverse populations. The value impact platform 120 systematically compares selection rates between predicted outcomes and training data, focusing on attributes including but not limited to gender, age, Area Deprivation Index (ADI) codes, Rural-Urban Commuting Area (RUCA) codes, and Social Determinants of Health (SDOH) Socioeconomic Status (SES) metrics. The fairness monitoring process identifies and mitigate biases, ensuring that the model's predictions do not disproportionately favor or disadvantage any group based on these sensitive features. In some embodiments, the value impact platform 120 includes one or more correction mechanisms in response to fairness modeling. In some embodiments, upon detection of bias or drift through the fairness monitoring component, the value impact platform 120 initiates corrective measures to adjust the model. These measures may include retraining the model with augmented datasets, applying algorithmic fairness techniques, or adjusting predictive thresholds. The platform is configured to automatically implement such corrections to ensure that model outputs remain in alignment with one or more predefined fairness criteria, such as equal opportunity, demographic parity, or the like.

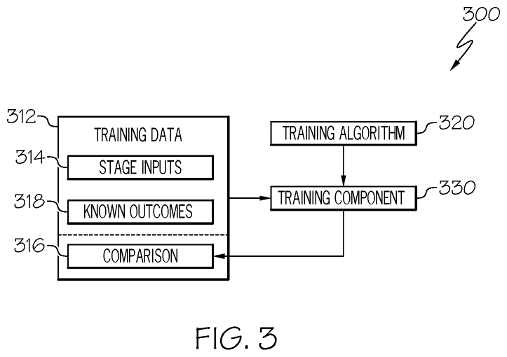

One or more implementations disclosed herein include and/or are implemented using a machine-learning model. For example, one or more of the modules of the value impact platform are implemented using a machine-learning model and/or are used to train the machine-learning model. FIG. 3 shows an example machine-learning training flow chart, according to some embodiments of the disclosure. Referring to FIG. 3, a given machine-learning model is trained using the training flow chart 300. The training data 312 includes one or more of stage inputs 314 and the known outcomes 318 related to the machine-learning model to be trained. The stage inputs 314 are from any applicable source including text, visual representations, data, values, comparisons, and stage outputs, e.g., one or more outputs from one or more steps from FIG. 2. The known outcomes 318 are included for the machine-learning models generated based on supervised or semi-supervised training, or can based on known labels, such as topic labels. An unsupervised machine-learning model is not trained using the known outcomes 318. The known outcomes 318 includes known or desired outputs for future inputs similar to or in the same category as the stage inputs 314 that do not have corresponding known outputs.

The training data 312 and a training algorithm 320, e.g., one or more of the modules implemented using the machine-learning model and/or are used to train the machine-learning model, is provided to a training component 330 that applies the training data 312 to the training algorithm 320 to generate the machine-learning model. According to an implementation, the training component 330 is provided comparison results 316 that compare a previous output of the corresponding machine-learning model to apply the previous result to re-train the machine-learning model. The comparison results 316 are used by the training component 330 to update the corresponding machine-learning model. The training algorithm 320 utilizes machine-learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine-learning model used herein is trained and/or used by adjusting one or more weights and/or one or more layers of the machine-learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the process illustrated in FIG. 2 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

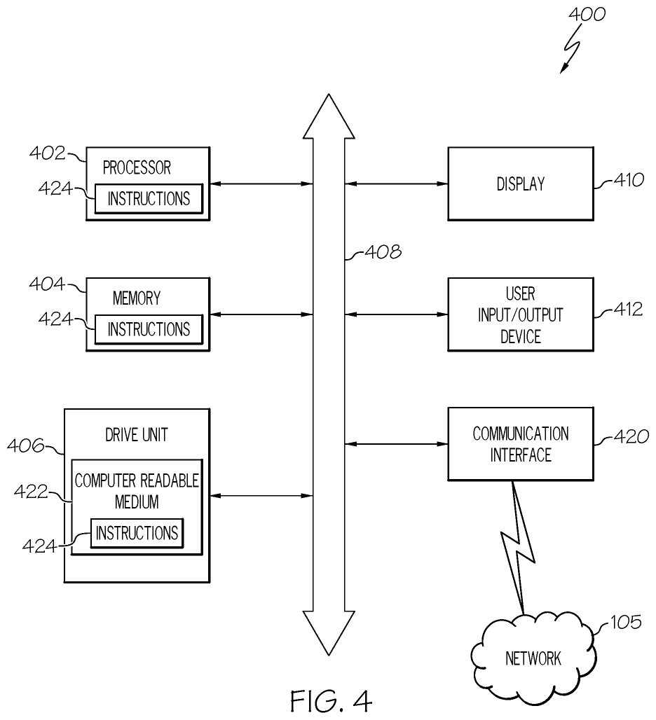

FIG. 4 illustrates an implementation of a computer system that executes techniques presented herein. The computer system 400 includes a set of instructions that are executed to cause the computer system 400 to perform any one or more of the methods or computer based functions disclosed herein. The computer system 400 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 400 operates in the capacity of a server or as a client user computer in a server-client user network environment, or as a peer computer system in a peer-to-peer (or distributed) network environment. The computer system 400 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 400 is implemented using electronic devices that provide voice, video, or data communication. Further, while the computer system 400 is illustrated as a single system, the term “system” shall also be taken to include any collection of systems or sub-systems that individually or jointly execute a set, or multiple sets, of instructions to perform one or more computer functions.

As illustrated in FIG. 4, the computer system 400 includes a processor 402, e.g., a central processing unit (CPU), a graphics processing unit (GPU), or both. The processor 402 is a component in a variety of systems. For example, the processor 402 is part of a standard personal computer or a workstation. The processor 402 is one or more processors, digital signal processors, application specific integrated circuits, field programmable gate arrays, servers, networks, digital circuits, analog circuits, combinations thereof, or other now known or later developed devices for analyzing and processing data. The processor 402 implements a software program, such as code generated manually (i.e., programmed).

The computer system 400 includes a memory 404 that communicates via bus 408. The memory 404 is a main memory, a static memory, or a dynamic memory. The memory 404 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 404 includes a cache or random-access memory for the processor 402. In alternative implementations, the memory 404 is separate from the processor 402, such as a cache memory of a processor, the system memory, or other memory. The memory 404 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts, or tasks illustrated in the figures or described herein are performed by the processor 402 executing the instructions stored in the memory 404. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 400 further includes a display 410, such as a liquid crystal display (LCD), an organic light emitting diode (OLED), a flat panel display, a solid-state display, a cathode ray tube (CRT), a projector, a printer or other now known or later developed display device for outputting determined information. The display 410 acts as an interface for the user to see the functioning of the processor 402, or specifically as an interface with the software stored in the memory 404 or in the drive unit 406.

Additionally or alternatively, the computer system 400 includes an input/output device 412 configured to allow a user to interact with any of the components of the computer system 400. The input/output device 412 is a number pad, a keyboard, a cursor control device, such as a mouse, a joystick, touch screen display, remote control, or any other device operative to interact with the computer system 400.

The computer system 400 also includes the drive unit 406 implemented as a disk or optical drive. The drive unit 406 includes a computer-readable medium 422 in which one or more sets of instructions 424, e.g. software, is embedded. Further, the sets of instructions 424 embodies one or more of the methods or logic as described herein. The sets of instructions 424 resides completely or partially within the memory 404 and/or within the processor 402 during execution by the computer system 400. The memory 404 and the processor 402 also include computer-readable media as discussed above.

In some systems, computer-readable medium 422 includes the set of instructions 424 or receives and executes the set of instructions 424 responsive to a propagated signal so that a device connected to network 105 communicates voice, video, audio, images, or any other data over the network 105. Further, the sets of instructions 424 are transmitted or received over the network 105 via the communication port or interface 420, and/or using the bus 408. The communication port or interface 420 is a part of the processor 402 or is a separate component. The communication port or interface 420 is created in software or is a physical connection in hardware. The communication port or interface 420 is configured to connect with the network 105, external media, the display 410, or any other components in the computer system 400, or combinations thereof. The connection with the network 105 is a physical connection, such as a wired Ethernet connection, or is established wirelessly as discussed below. Likewise, the additional connections with other components of the computer system 400 are physical connections or are established wirelessly. The network 105 alternatively be directly connected to the bus 408.

While the computer-readable medium 422 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 422 is non-transitory, and may be tangible.

The computer-readable medium 422 includes a solid-state memory such as a memory card or other package that houses one or more non-volatile read-only memories. The computer-readable medium 422 is a random-access memory or other volatile re-writable memory. Additionally or alternatively, the computer-readable medium 422 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 400 is connected to the network 105. The network 105 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 105 includes wide area networks (WAN), such as the Internet, local area networks (LAN), campus area networks, metropolitan area networks, a direct connection such as through a Universal Serial Bus (USB) port, or any other networks that allows for data communication. The network 105 is configured to couple one computing device to another computing device to enable communication of data between the devices. The network 105 is generally enabled to employ any form of machine-readable media for communicating information from one device to another. The network 105 includes communication methods by which information travels between computing devices. The network 105 is divided into sub-networks. The sub-networks allow access to all of the other components connected thereto or the sub-networks restrict access between the components. The network 105 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the disclosure requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the instructions for carrying out such a method or element of a method forms a means for carrying out the method or element of a method. Furthermore, an element described herein of an apparatus embodiment is an example of a means for carrying out the function performed by the element for the purpose of carrying out the disclosure.

In the description provided herein, numerous specific details are set forth. However, it is understood that embodiments of the disclosure are practiced without these specific details. In other instances, well-known methods, structures and techniques have not been shown in detail in order not to obscure an understanding of this description.

Thus, while there has been described what are believed to be the preferred embodiments of the disclosure, those skilled in the art will recognize that other and further modifications are made thereto without departing from the spirit of the disclosure, and it is intended to claim all such changes and modifications as falling within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects:

Example 1. A computer-implemented method comprising: receiving, by one or more processors, a first data object, the first data object including: an entity data set containing a plurality of entities; a utilization event data set containing a plurality of utilization event records; an event data set; an environmental data set; and a performance metric data set; generating, by the one or more processors, an entity data object for each entity of the plurality of entities based on at least one of the entity data set, the utilization event data set, the event data set, the environmental data set, or the performance metric data set; generating, by the one or more processors, a usage indicator for each entity of the plurality of entities by applying a machine-learning model to at least a portion of the entity data object for the entity, the usage indicator associated with a pre-determined time period; generating, by the one or more processors, a utilization data object based on the entity data object and the usage indicator generated for each entity; and causing, by the one or more processors, the utilization data object to be displayed on a Graphical User Interface (GUI).

Example 2. The computer-implemented method of example 1, wherein the machine-learning model is trained to identify associations between the entity data objects and respective probabilities of internal system utilization during the pre-determined time period.

Example 3. The computer-implemented method of Example 2, wherein the internal system utilization includes at least one of emergency resource utilization or internal services utilization, and the machine-learning model is trained based at least in part on based on i) training data set associated with a plurality of sample entities, the training data set including at least one of a sample entity data set, a sample utilization event data set, a sample event data set, a sample environmental data set, or a sample performance metric data set, and ii) respective probabilities of internal system utilization associated with the plurality of sample entities.

Example 4. The computer-implemented method of any of Examples 1-3, further comprising: associating, by the one or more processors, the usage indicator for each entity with a risk score, the risk score indicative of a probability of internal system utilization by the respective entity during the pre-determined period of time.

Example 5. The computer-implemented method of any of Examples 1-4, wherein the utilization data object is generated using one or more classification codes from the performance metric data set, the classification codes identifying one or more preventable utilization events.

Example 6. The computer-implemented method of Example 5 further comprising: determining, by the one or more processors, an intervention action based on the utilization data object for each entity, wherein the intervention action is targeted to mitigate a likelihood of the respective entity's one or more preventable utilization events.

Example 7. The computer-implemented method of Example 6 further comprising: calculating, by the one or more processors, a resource efficiency metric for each entity based on at least one of the utilization data object, the usage indicator, the identified one or more preventable utilization events, or the determined intervention action.

Example 8. The computer-implemented method of Example 7, wherein the resource efficiency metric is further based on at least one of historical costs and visit data for the entity or historical costs and visit data for one or more additional entities.

Example 9. The computer-implemented method of Example 8, wherein the determined intervention action includes administration of one or more preventative actions to the respective entity associated with the utilization data object.

Example 10. The computer-implemented method of any of Examples 1-9, further comprising: determining, by the one or more processors and using a scenario modeling technique, one or more possible effects of the determined intervention action on internal system utilization or intervention efficacy.

Example 11. A system comprising memory and one or more processors communicatively coupled to the memory, the one or more processors configured to: receive a first data object, the first data object including: an entity data set containing a plurality of entities; a utilization event data set containing a plurality of utilization event records; an event data set; an environmental data set; and a performance metric data set; generate an entity data object for each entity of the plurality of entities based on at least one of the entity data set, the utilization event data set, the event data set, the environmental data set, or the performance metric data set; generate a usage indicator for each entity of the plurality of entities by applying a machine-learning model to at least a portion of the entity data object for the entity, the usage indicator associated with a pre-determined time period; generate a utilization data object based on the entity data object and the usage indicator generated for each entity; and cause the utilization data object to be displayed on a Graphical User Interface (GUI).

Example 12. The system of Example 11, wherein the machine-learning model is trained to identify associations between the entity data objects and respective probabilities of internal system utilization during the pre-determined time period.

Example 13. The system of Example 12, wherein the internal system utilization includes at least one of emergency resource utilization or internal services utilization, and the machine-learning model is trained based at least in part on based on i) training data set associated with a plurality of sample entities, the training data set including at least one of a sample entity data set, a sample utilization event data set, a sample event data set, a sample environmental data set, or a sample performance metric data set, and ii) respective probabilities of internal system utilization associated with the plurality of sample entities.

Example 14. The system of any of Examples 10-13, the one or more processors further configured to associate the usage indicator for each entity with a risk score, the risk score indicative of a probability of internal system utilization by the respective entity during the pre-determined period of time.

Example 15. The system of any of Examples 10-14, wherein the utilization data object is generated using one or more classification codes from the performance metric data set, the classification codes identifying one or more preventable utilization events.

Example 16. The system of Example 15, the one or more processors further configured to determine an intervention action based on the utilization data object for each entity, wherein the intervention action is targeted to mitigate a likelihood of the respective entity's one or more preventable utilization events.

Example 17. The system of Example 16, the one or more processors further configured to calculate a resource efficiency metric for each entity based on at least one of the utilization data object, the usage indicator, the identified one or more preventable utilization events, or the determined intervention action.

Example 18. The system of Example 17, wherein the resource efficiency metric is further based on at least one of historical costs and visit data for the entity or historical costs and visit data for one or more additional entities.

Example 19. The system of Example 18, wherein the determined intervention action includes administration of one or more preventative actions to the respective entity associated with the utilization data object.

Example 20. One or more non-transitory computer-readable storage media including instructions that, when executed by one or more processors, cause the one or more processors to: receive a first data object, the first data object including: an entity data set containing a plurality of entities; a utilization event data set containing a plurality of utilization event records; an event data set; an environmental data set; and a performance metric data set; generate an entity data object for each entity of the plurality of entities based on at least one of the entity data set, the utilization event data set, the event data set, the environmental data set, or the performance metric data set; generate a usage indicator for each entity of the plurality of entities by applying a machine-learning model to at least a portion of the entity data object for the entity, the usage indicator associated with a pre-determined time period; generate a utilization data object based on the entity data object and the usage indicator generated for each entity; and cause the utilization data object to be displayed on a Graphical User Interface (GUI).

What is claimed is:

1. A computer-implemented method comprising:
receiving, by one or more processors, a first data object, the first data object including:
an entity data set containing a plurality of entities;
a utilization event data set containing a plurality of utilization event records;
an event data set;
an environmental data set; and
a performance metric data set;
generating, by the one or more processors, an entity data object for each entity of the plurality of entities based on at least one of the entity data set, the utilization event data set, the event data set, the environmental data set, or the performance metric data set;
generating, by the one or more processors, a usage indicator for each entity of the plurality of entities by applying a machine-learning model to at least a portion of the entity data object for the entity, the usage indicator associated with a pre-determined time period;
generating, by the one or more processors, a utilization data object based on the entity data object and the usage indicator generated for each entity; and
causing, by the one or more processors, the utilization data object to be displayed on a Graphical User Interface (GUI).

2. The computer-implemented method of claim 1, wherein the machine-learning model is trained to identify associations between the entity data objects and respective probabilities of internal system utilization during the pre-determined time period.

3. The computer-implemented method of claim 2, wherein the internal system utilization includes at least one of emergency resource utilization or internal services utilization, and the machine-learning model is trained based at least in part on based on i) training data set associated with a plurality of sample entities, the training data set including at least one of a sample entity data set, a sample utilization event data set, a sample event data set, a sample environmental data set, or a sample performance metric data set, and ii) respective probabilities of internal system utilization associated with the plurality of sample entities.

4. The computer-implemented method of claim 1, further comprising: associating, by the one or more processors, the usage indicator for each entity with a risk score, the risk score indicative of a probability of internal system utilization by the respective entity during the pre-determined period of time.

5. The computer-implemented method of claim 1, wherein the utilization data object is generated using one or more classification codes from the performance metric data set, the classification codes identifying one or more preventable utilization events.

6. The computer-implemented method of claim 5 further comprising: determining, by the one or more processors, an intervention action based on the utilization data object for each entity, wherein the intervention action is targeted to mitigate a likelihood of the respective entity's one or more preventable utilization events.

7. The computer-implemented method of claim 6 further comprising: calculating, by the one or more processors, a resource efficiency metric for each entity based on at least one of the utilization data object, the usage indicator, the identified one or more preventable utilization events, or the determined intervention action.

8. The computer-implemented method of claim 7, wherein the resource efficiency metric is further based on at least one of historical costs and visit data for the entity or historical costs and visit data for one or more additional entities.

9. The computer-implemented method of claim 8, wherein the determined intervention action includes administration of one or more preventative actions to the respective entity associated with the utilization data object.

10. The computer-implemented method of claim 1, further comprising: determining, by the one or more processors and using a scenario modeling technique, one or more possible effects of the determined intervention action on internal system utilization or intervention efficacy.

11. A system comprising memory and one or more processors communicatively coupled to the memory, the one or more processors configured to:
receive a first data object, the first data object including:
an entity data set containing a plurality of entities;
a utilization event data set containing a plurality of utilization event records;
an event data set;
an environmental data set; and
a performance metric data set;
generate an entity data object for each entity of the plurality of entities based on at least one of the entity data set, the utilization event data set, the event data set, the environmental data set, or the performance metric data set;
generate a usage indicator for each entity of the plurality of entities by applying a machine-learning model to at least a portion of the entity data object for the entity, the usage indicator associated with a pre-determined time period;
generate a utilization data object based on the entity data object and the usage indicator generated for each entity; and
cause the utilization data object to be displayed on a Graphical User Interface (GUI).

12. The system of claim 11, wherein the machine-learning model is trained to identify associations between the entity data objects and respective probabilities of internal system utilization during the pre-determined time period.

13. The system of claim 12, wherein the internal system utilization includes at least one of emergency resource utilization or internal services utilization, and the machine-learning model is trained based at least in part on based on i) training data set associated with a plurality of sample entities, the training data set including at least one of a sample entity data set, a sample utilization event data set, a sample event data set, a sample environmental data set, or a sample performance metric data set, and ii) respective probabilities of internal system utilization associated with the plurality of sample entities.

14. The system of claim 11, the one or more processors further configured to associate the usage indicator for each entity with a risk score, the risk score indicative of a probability of internal system utilization by the respective entity during the pre-determined period of time.

15. The system of claim 11, wherein the utilization data object is generated using one or more classification codes from the performance metric data set, the classification codes identifying one or more preventable utilization events.

16. The system of claim 15, the one or more processors further configured to determine an intervention action based on the utilization data object for each entity, wherein the intervention action is targeted to mitigate a likelihood of the respective entity's one or more preventable utilization events.

17. The system of claim 16, the one or more processors further configured to calculate a resource efficiency metric for each entity based on at least one of the utilization data object, the usage indicator, the identified one or more preventable utilization events, or the determined intervention action.

18. The system of claim 17, wherein the resource efficiency metric is further based on at least one of historical costs and visit data for the entity or historical costs and visit data for one or more additional entities.

19. The system of claim 18, wherein the determined intervention action includes administration of one or more preventative actions to the respective entity associated with the utilization data object.

20. One or more non-transitory computer-readable storage media including instructions that, when executed by one or more processors, cause the one or more processors to:
receive a first data object, the first data object including:
an entity data set containing a plurality of entities;
a utilization event data set containing a plurality of utilization event records;
an event data set;
an environmental data set; and
a performance metric data set;
generate an entity data object for each entity of the plurality of entities based on at least one of the entity data set, the utilization event data set, the event data set, the environmental data set, or the performance metric data set;
generate a usage indicator for each entity of the plurality of entities by applying a machine-learning model to at least a portion of the entity data object for the entity, the usage indicator associated with a pre-determined time period;
generate a utilization data object based on the entity data object and the usage indicator generated for each entity; and
cause the utilization data object to be displayed on a Graphical User Interface (GUI).
